(19) United States
(12) Patent Application Publication
(10) Pub. No.: US 2022/0143179 A1
(43) Pub. Date: May 12, 2022

(54) DRUG DELIVERY FOR COMBINATION OF EPIGENETIC MODULATION AND IMMUNE CHECKPOINT BLOCKADE

(71) Applicant: NORTH CAROLINA STATE UNIVERSITY, Raleigh, NC (US)

(72) Inventors: Zhen GU, Los Angeles, CA (US); Huitong RUAN, Raleigh, NC (US); Quanyin HU, Raleigh, NC (US)

(21) Appl. No.: 17/436,146

(22) PCT Filed: Mar. 4, 2020
(86) PCT No.: PCT/US2020/020853

§ 371 Date: Sep. 3, 2021

Related U.S. Application Data
(60) Provisional application No. 62/813,442, filed on Mar. 4, 2019.

(51) Int. Cl. A61K 39/395; A61K 9/06; C07K 16/28; A61K 31/7068; A61K 31/706; A61K 31/353; A61K 31/245; A61K 31/405; A61K 31/711; A61K 31/7084; A61K 47/32; A61K 47/24; A61P 35/00

(52) U.S. Cl. CPC A61K 39/3955; A61P 35/00; C07K 16/2818; C07K 16/2827; C07K 16/2803; A61K 31/7068; A61K 31/706; A61K 31/353; A61K 31/245; A61K 31/405; A61K 31/711; A61K 31/7084; A61K 47/32; A61K 47/24; A61K 9/06

(57) ABSTRACT

Disclosed are compositions comprising dual bioresponsive hydrogels and methods of their use.

This application claims the benefit of U.S. Provisional Application No. 62/813,442, filed on Mar. 4, 2019, which is incorporated herein by reference in its entirety.

### I. BACKGROUND

Programmed death-1 (PD-1) receptor is expressed on various immune cells, including activated CD8⁺ T cells. The interaction between PD-1 and its ligand PD-L1/PD-L2 on tumor cells such as melanoma cells can lead to T cell anergy, impeding anticancer immune responses. Therefore, blocking PD-1/PD-L1 pathway by anti-PD-1 (aPD1) or anti-PD-L1 antibodies (aPDL1) can revert the exhausted T cells and enhance anti-tumor immune responses in patients with melanoma or other cancers. However, despite the considerable success of PD-1/PD-L1 blockade therapy, utilization of these antibodies as a single therapeutic is often limited to a subset of patients. Several immune evasion mechanisms account for it, including the absence of tumor-associated antigens (TAAs), infiltration of immunosuppressive cells, such as myeloid-derived suppressor cells (MDSCs) and regulatory T cells (Tregs). What are needed are new methods and compositions for reducing T cell exhaustion by immunosuppressive pathways.

### II. SUMMARY

Disclosed are methods and compositions related to bioresponsive hydrogels.

In one aspect, disclosed herein are bioresponsive hydrogels comprising a first therapeutic agent and an engineered particle, wherein the engineered particle comprises a second therapeutic agent.

Also disclosed herein are bioresponsive hydrogels of any preceding aspect, wherein one of the first therapeutic agent or the second therapeutic agent comprises a blockade inhibitor (such as, for example, PD-1/PD-L1, CTLA-4/B7-1/2, and/or CD47/SIRPα inhibitors) and the remaining therapeutic agent comprises a hypomethylatingagent (HMA) (such as, for example, Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine).

In one aspect, disclosed herein are bioresponsive hydrogels of any preceding aspect, wherein the bioresponsive hydrogel comprises a bioresponsive scaffold that releases the first therapeutic agent and the engineered particle into a tumor microenvironment upon exposure to factors within the microenvironment (such as, for example, a reactive oxygen species (ROS) degradable hydrogel). In one aspect, the hydrogel can comprise crosslinked polyvinyl alcohol (PVA) and N¹-(4-boronobenzyl)-N³-(4-boronophenyl)-N¹,N¹,N³,N³-tetramethylpropane-1,3-diaminium (TSPBA).

Also disclosed herein are bioresponsive hydrogels of any preceding aspect, wherein the engineered particles comprise a pH responsive material (such as, for example, dextran, CaCO3, chitosan, hyaluronic acid, as well as polymers thereof including, for example polymers of dextran monomers (for example a polymer of m-dextran monomers).

In one aspect, disclosed herein are methods of treating a cancer in a subject comprising administering to the subject the bioresponsive hydrogels of any preceding aspect.

In one aspect, disclosed herein are methods of treating a cancer in a subject comprising administering to the subject a bioresponsive hydrogel and an engineered particle; wherein the bioresponsive hydrogel comprises a first therapeutic agent; and wherein the particle comprises a second therapeutic agent. In one aspect, the bioresponsive hydrogel and engineered particle are administered concurrently. In one aspect, the engineered particle comprising the second therapeutic agent is encapsulated in the bioresponsive hydrogel.

Also disclosed herein are methods of treating a cancer of any preceding aspect, wherein the hydrogel releases the first therapeutic agent and/or the engineered particle into the tumor microenvironment for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In one aspect, the first therapeutic agent and the engineered particle are released from the hydrogel at the same or different rates.

In one aspect, disclosed herein are methods of treating a cancer in a subject, further comprising administering to the subject an anti-cancer agent different than both the first and second therapeutic agent. In some aspect, the anti-cancer agent can be comprised in the bioresponsive hydrogel.

Also disclosed are methods treating a cancer of any preceding aspect, wherein the cancer comprises a cancer with low PD-L1 expression or a non-immunogenic cancer selected from the group consisting of melanoma, non-small cell lung carcinoma, renal cancer, head and neck cancer, and bladder cancer.

Also disclosed herein are methods of inducing blockade inhibitor susceptibility in a tumor in a subject with a cancer comprising administering to the subject a bioresponsive hydrogel of any preceding aspect (such as, for example, a bioresponsive hydrogel comprising a first therapeutic agent and an engineered particle, wherein the engineered particle comprises a second therapeutic agent; and wherein one of the therapeutic agent comprises a hypomethylating agent (HMA) and the other therapeutic agent comprises an immune blockade inhibitor).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, and 1F show schematic and characterization of injectable in situ formed ROS/H⁺ dual bioresponsive gel depots. FIG. 1A shows schematic illustrating the combination strategy of epigenetic modulation and immune checkpoint blockade (ICB) therapy using ROS/H⁺ responsive scaffolds. FIG. 1B shows size distribution of aPD1-loaded CaCO₃ nanoparticles (aPD1-NPs) measured by dynamic light scattering (DLS) and morphology observation by transmission electronic microscopy (TEM). Scale bar: 100 nm. FIG. 1C shows representative cryo-scanning electron microscopy (Cyro-SEM) image of hydrogel loaded with aPD1-NPs. Scale bar: 500 nm. FIG. 1D shows release profiles of Zeb from hydrogel incubated in PBS buffer (pH 7.4) with or without 1 mM H₂O₂. FIG. 1E shows release profiles of aPD1 from NPs-loaded gel depot incubated in PB buffer (pH 7.4 or pH 6.5) with/without 1 mM H₂O₂. The data are presented as mean±SD, n=3. FIG. 1F shows In vivo retention of Cy5.5-labeled aPD1 in different formulations at different days (day 0 (D0), day 2 (D2), day 4 (D4), day 6 (D6)), injected at peritumoral sites in the B16F10 melanoma-bearing mice (G1: free Cy5.5-aPD1; G2: Cy5.5-aPD1-NPs; G3: Cy5.5-aPD1-NPs-Gel).

FIG. 2 shows characterization of H₂O₂-labile TSPBA linker by 41-NMR (300 MHz, in D20).

FIG. 3 shows the release profiles of aPD1 from CaCO₃ NPs in PB buffers at different pH values (pH 6.5 or pH 7.4). The data are presented as mean±SD (n=3).

FIGS. 4A, 4B, 4C, 4D, 4E, 4F, and 4G show ROS-responsive Zeb-Gel incorporation for regulating tumor immunogenicity and immunosuppressive tumor microenvironmentin vivo. The tumors were collected from subcutaneous B16F10 melanoma-bearing mice treated with Zeb-Gel (5 mg/kg) at the peritumoral sites for five days. UnTx reprented untreated group. FIG. 4A shows TAAs expression including MAGE-E1, TRP1, and CD146 analyzed by western blotting assay. FIGS. 4B and 4C show quantitative analysis of PD-L1 expression of tumor cells by flow cytometry. FIGS. 4D and 4E show representative images and the quantitative analysis of dendritic cells (DCs) (CD80⁺CD86⁺) gating on CD11c⁺ cells by flow cytometry. The data are presented as mean±SD, n=3, **p<0.01. FIGS. 4F and 4G show representative images and the quantitative analysis of MDSCs (CD11b⁺Gr-1⁺) gating on CD45⁺ cells by flow cytometry. The data are presented as mean±SD, n=3, *p<0.05.

FIGS. 5A, 5B, 5C, 5D, and 5E show combination therapy for the treatment of in vivo B16F10 melanoma-bearing mice by enhancing anti-tumor immune response. FIG. 5A shows representative in vivo bioluminescence images of mice treated with different formulations at different time points (D0, D4, D8), including blank Gel, aPD1-NPs-Gel (aPD1, 40 μg per mouse), aPD1-NPs-Gel+Zeb (aPD1, 40 μg per mouse; Zeb, 5 mg/kg), Zeb-NPs-Gel (Zeb, 5 mg/kg), and Zeb-aPD1-NPs-Gel (aPD1, 40 μg per mouse; Zeb, 5 mg/kg). FIG. 5B shows tumor growth curves of subcutaneous B16F10 tumor in C57BL/6 mice receiving various formulations. (day 0 represents the treatment-receiving day). The data are presented as mean±SD, n=6, ***p<0.001. FIG. 5C shows Kaplan-Meier survival curves of model mice (n=6). The survival curve of Zeb-NPs-Gel group was identical with that of UnTx group. Statistical significance was calculated via the Log-Rank test, ***p<0.001. FIG. 5D shows quantitative analysis of the percentage of CD8⁺ T cells in tumor tissues, which were harvested five days after local delivery of different formulations. The data are presented as mean±SD, n=3. One-way ANOVA with Tukey's post hoc test, **p<0.01, ***p<0.001. FIG. 5E shows representative images of CD4⁺ and CD8⁺ T cells gated on CD3⁺ T cells by flow cytometry.

FIGS. 6A and 6B shows representative flow cytometric analysis images and relative quantification of CD8⁺CD44⁺ T cells gating on CD8⁺ cells in the group treated with different formulations. FIG. 6A shows representative flow cytometric analysis images of CD8⁺CD44⁺ T cells gating on CD8⁺ cells in the group treated with different formulations. FIG. 6B shows quantitative analysis of the percentage of CD8⁺CD44⁺ T cells in tumor tissues. The data are presented as mean±SD (n=3). One-way ANOVA with Tukey's post hoc test, *p<0.05, **p<0.01.

FIGS. 7A, 7B, 7C, 7D, and 7E show the systemic immune responses after local delivery of Zeb-aPD1-NPs-Gel. B16F10 cells were implanted in mice on both sides and only the tumors on the left side were treated with Zeb-aPD1-NPs-Gel (aPD1, 40 μg per mouse; Zeb, 5 mg/kg). FIG. 7A shows In vivo bioluminescence imaging of model mice at different days (treatment started at day 0). FIG. 7B shows tumor growth curves on both sides. The data are presented as mean±SD, n=5, ***p<0.001. FIGS. 7C, 7D, and 7E show representative flow cytometric images and quantitative analysis of CD4⁺ and CD8⁺ T cells in tumor cells ten days after treatment. The data are presented as mean±SD, n=3. One-way ANOVA with Tukey's post hoc test, *p<0.05.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular bioresponsive hydrogel and/or engineered particle is disclosed and discussed and a number of modifications that can be made to a number of molecules including the bioresponsive hydrogel and/or engineered particle are discussed, specifically contemplated is each and every combination and permutation of bioresponsive hydrogel and/or engineered particle and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Epigenetic alteration like DNA hypermethylation plays a pivotal role in immune evasion during tumorigenesis. It is a common feature of heterogeneous cancer phenotypes for the reason that the TAA promoter regions have been detected to be hypermethylated in various types of cancers. It has been reported that hypomethylating agents (HMAs), also known as DNA methyltransferase inhibitor (DNMTi), could contribute to enhancing the expression of TAAs, which increase tumor immunogenicity and enhance infiltration of CD8⁺ T cells. Additionally, DNA methylation is involved in alerting host immune responses as well, and HMAs have been demonstrated to regulate immunosuppressive tumor microenviroment by reducing MDSCs. It is shown herein that HMAs also induced expression of immunosuppressive ligands, such as PD-L1/PD-L2, sensitizing tumors to PD-1/PD-L1 checkpoint blockade therapy.

Disclosed herein are bioresponsive hydrogels and engineered particles; wherein the bioresponsive hydrogel comprises a first therapeutic agent and the engineered particle comprises a second therapeutic agent. In one aspect, the engineered particles can be encapsulated in the bioresponsive hydrogel. Thus, in one aspect, disclosed herein are bioresponsive hydrogels comprising a first therapeutic agent and an engineered particle; wherein the engineered particle comprises a second therapeutic agent.

As noted above, he hydrogels and/or engineered particles disclosed herein are designed to sensitize a subject with a cancer to immune blockade inhibition therapy. Thus, in one aspect, it is understood and herein contemplated that either the first or second therapeutic agent comprises a blockade inhibitor (such as, for example, PD-1/PD-L1, CTLA-4/B7-1/2, and/or CD47/SIRPα inhibitors) and the remaining therapeutic agent comprises a hypomethylating agent (HMA) (such as, for example, Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine).

In one aspect, the blockade inhibitor that can be used in the disclosed bioresponsive hydrogels and/or engineered particles can be any inhibitor of an immune checkpoint blockade inhibitor, such as for example, a PD-1/PD-L1 blockade inhibitor, a CTLA-4/B7-1/2 blockade inhibitor (such as for example, Ipilimumab), and CD47/Signal Regulator Protein alpha (SIRPα) blockade inhibitor (such as for example, Hu5F9-G4, CV1, B6H12, 2D3, CC-90002, and/or TTI-621). Examples, of PD-1/PD-L1 blockade inhibitors for use in the disclosed bioresponsive hydrogels can include any PD-1/PD-L1 blockade inhibitor known in the art, including, but not limited to nivolumab, pembrolizumab, pidilizumab, atezolizumab, avelumab, durvalumab, and BMS-936559).

As noted herein, the disclosed bioresponsive hydrogels and/or engineered particles utilize an HMA that is either embedded in the bioresponsive hydrogel or integrated in the engineered particle (the particle which itself can be encapsulated in the bioresponsive hydrogel) to sensitize the subject to immune checkpoint inhibition therapy. It is understood and herein contemplated that the HMA used in the disclosed hydrogels and/or engineered particles can comprise any known HMA, including, but not limited to Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine. Thus, in one aspect, disclosed herein are any bioresponsive hydrogel and/or engineered particle disclosed herein (including hydrogels comprising a first therapeutic agent and a engineered particle embedded in the hydrogel matrix); wherein either the first therapeutic agent or a second therapeutic agent comprised by the engineered particle comprises an HMA; and wherein the HMA comprises Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine.

To facilitate these functions, the bioresponsive hydrogel can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the bioresponsive hydrogel can comprise a biocompatible polymer (such as, for example, methacrylated hyaluronic acid (m-HA)). In one aspect, biocompatible polymer can be crosslinked. Such polymers can also serve to slowly release the adipose browning agent and/or fat modulating agent into tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like. In one aspect, the core polymer can be flanked by polypeptide blocks.

Examples of diblock copolymers that can be used in the micelles disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA)

It is understood and herein contemplated that the bioresponsive hydrogel can be designed to be bioresponsive to the microenvironment of the tumor and release the first therapeutic agent, any encapsulated engineered particle (including engineered particles comprising a second therapeutic agent), and any further anti-cancer agents into the tumor microenvironment upon exposure to factors within the microenvironment such as, for example reactive oxygen species, including, but not limited to peroxides (for example hydrogen peroxide), superoxide, hydroxyl radical, and singlet oxygen; the presence of acidity; redox potential (glutathione (GSH)); specific tumor-associated enzymes; hypoxia; and adenosine-5′-triphosphate (ATP). Thus, in one aspect, disclosed herein the bioresponsive hydrogels disclosed herein comprises a bioresponsive scaffold that releases the first therapeutic agent and any encapsulated engineered particle, and/or further anti-cancer agent into a tumor microenvironment upon exposure to factors within the microenvironment (such as, for example, a reactive oxygen species (ROS) degradable hydrogel). In one aspect, the hydrogel can comprise crosslinked polyvinyl alcohol (PVA) and N¹-(4-boronobenzyl)-N³-(4-boronophenyl)-N¹,N¹,N³,N³-tetramethylpropane-1,3-diaminium (TSPBA). In one aspect, the ROS-responsive hydrogel can be obtained by crosslinking poly (vinyl alcohol) (PVA) with a ROS-labile linker: N¹-(4-boronobenzyl)-N³-(4-boronophenyl)-N¹,N¹,N³,N³-tetramethylpropane-1,3-diaminium (TSPBA), which was synthesized via quaternization reaction of N¹,N¹,N³, N³-tetramethylpropane-1,3-diamine with an excess of 4-(bromomethyl) phenylboronic acid. TSPBA contains two phenylboronic acids that complex with multiple diols on PVA. The TSPBA can be oxidized and hydrolyzed when exposed to H₂O₂ in the tumor microenvironment, leading to the dissociation of the polymeric scaffold and the release of PVA and payloads.

In one aspect, it is understood and herein contemplated that the disclosed engineered particles and/or bioresponsive hydrogels can also be responsive to the pH in the tumor microenvironment. In one aspect, disclosed herein are engineered particles (for example nanoparticles) comprising a second therapeutic agent, as well, as bioresponsive hydrogels comprising said engineered particles; wherein the engineered particles comprise a pH responsive material (such as, for example, dextran, CaCO3, chitosan, hyaluronic acid, as well as polymers thereof including, for example polymers of dextran monomers (for example a polymer of m-dextran monomers).

In one aspect, disclosed herein the bioresponsive hydrogels can comprise more than one nanoparticle. For example, the bioresponsive hydrogel can comprise 2, 3, 4, 5, 6, 7, 8, 9, or 10 nanoparticles. It is further understood and herein contemplated that the nanoparticles can comprise more than one type of anti-cancer agent, blockade inhibitor, or HMA. For example, the engineered particle (such as a nanoparticle) can comprise any combination of 1, 2, 3, 4, 5, 6, 7, 8, 910, 11, 12, 13, 14, 15, 16, 17, 18, 19, or 20 anti-cancer agents, blockade inhibitors, or HMAs. Additionally, it is understood and herein contemplated that in additional to the one or more anti-cancer agents, blockade inhibitors, or HMAs incorporated in the engineered particle, the bioresponsive hydrogel can also comprise an additional 1, 2, 3, 4, 5, 6, 7, 8, 9, 10, 11, 12, 13, 14, 15, 16, 17, 18, 19, or 20 anti-cancer agents, blockade inhibitors, HMAs, or engineered particles.

Anti-cancer agents that can be used in the disclosed bioresponsive hydrogels can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

### 1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof are also disclosed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen. 63. As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Method of Treating Cancer

In one aspect, disclosed herein are methods of treating, preventing, inhibiting, or reducing a cancer or metastasis (such as, for example, a cancer with low PD-L1 expression or a non-immunogenic cancer selected from the group consisting of melanoma, non-small cell lung carcinoma, renal cancer, head and neck cancer, and/or bladder cancer) in a subject comprising administering to the subject any of the bioresponsive hydrogels disclosed herein. For example, disclosed herein are methods of treating, preventing, inhibiting, or reducing a cancer or metastasis (such as, for example, a cancer with low PD-L1 expression or a non-immunogenic cancer selected. from the group consisting of melanoma, non-small cell lung carcinoma, renal cancer, head and neck cancer, and/or bladder cancer) in a subject comprising administering to the subject a bioresponsive hydrogel and an engineered particle; wherein the bioresponsive hydrogel comprises a first therapeutic agent; and wherein the particle comprises a second therapeutic agent.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

In one aspect, either the first therapeutic agent or the second therapeutic agent used in the disclosed methods of treating, preventing, inhibiting, or reducing a cancer or metastasis in a subject comprises a blockade inhibitor and the remaining therapeutic agent comprises a hypomethylating agent (HMA). In one aspect, the blockade inhibitor that can be used in the disclosed methods can be any inhibitor of an immune checkpoint such as for example, a PD-1/PD-L1 blockade inhibitor, a CTLA-4/B7-1/2 blockade inhibitor (such as for example, Ipilimumab), and CD47/Signal Regulator Protein alpha (SIRPα) blockade inhibitor (such as for example, Hu5F9-G4, CV1, B6H12, 2D3, CC-90002, and/or TTI-621). Examples, of PD-1/PD-L1 blockade inhibitors for use in the disclosed bioresponsive hydrogels can include any PD-1/PD-L1 blockade inhibitor known in the art, including, but not limited to nivolumab, pembrolizumab, pidilizumab, atezolizumab, avelumab, durvalumab, and BMS-936559). Thus, in one aspect, disclosed herein are methods of treating, preventing, inhibiting, or reducing a cancer or metastasis in a subject comprising administering to the subject a bioresponsive hydrogel and an engineered particle; wherein the bioresponsive hydrogel comprises a first therapeutic agent; and wherein the particle comprises a second therapeutic agent; wherein, either the first therapeutic agent or the second therapeutic agent comprises a blockade inhibitor; wherein the blockade inhibitor is a PD-1/PD-L1 blockade inhibitor such as, for example, nivolumab, pembrolizumab, pidilizumab, atezolizumab, avelumab, durvalumab, and BMS-936559; a CTLA-4/B7-1/2 inhibitor such as, for example, Ipilimumab; and/or a CD47/SIRPα inhibitor such as, for example Hu5F9-G4, CV1, B6H12, 2D3, CC-90002, and TTI-621. It is understood and herein contemplated that the bioresponsive hydrogel can be designed to incorporate 1, 2, 3, 4, 5, 6, 7, 8, 9, 10, 11, 12, 13, 14, or 15 blockade inhibitors simultaneously.

As noted herein, the disclosed methods utilize an HMA that is either embedded in the bioresponsive hydrogel or integrated in the engineered particle (the particle which itself can be encapsulated in the bioresponsive hydrogel). It is understood and herein contemplated that the HMA used in the disclosed methods can comprise any known HMA, including, but not limited to Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine. Thus, in one aspect, disclosed herein are methods of treating, preventing, inhibiting, or reducing a cancer or metastasis in a subject comprising administering to the subject a bioresponsive hydrogel and an engineered particle; wherein the bioresponsive hydrogel comprises a first therapeutic agent; and wherein the particle comprises a second therapeutic agent; wherein either the first therapeutic agent or the second therapeutic agent comprises a HMA; and wherein the HMA comprises Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine.

In one aspect, the disclosed methods of treating, preventing, inhibiting, or reducing a cancer or metastasis comprising administering to a subject any of the therapeutic agent delivery vehicles or pharmaceutical compositions disclosed herein (such as the disclosed engineered particles and bioresponsive hydrogels, including but not limited to bioresponsive hydrogels comprising the engineered particles) can comprise administration of the pharmaceutical compositions, bioresponsive hydrogels, and/or engineered particles at any frequency appropriate for the treatment of the particular cancer in the subject. For example, pharmaceutical compositions, bioresponsive hydrogels, and/or engineered particles can be administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months. In one aspect, the pharmaceutical compositions, bioresponsive hydrogels, and/or engineered particles are administered at least 1, 2, 3, 4, 5, 6, 7 times per week.

As disclosed herein the bioresponsive hydrogel scaffold can be designed to release any therapeutic agent, engineered particle, or additional anti-cancer agent encapsulated in the hydrogel as the degradation of the hydrogel occurs in response to factors in the tumor microenvironment. Accordingly disclosed herein are methods of treating, preventing, inhibiting, or reducing a cancer or metastasis in a subject wherein the bioresponsive hydrogel comprises a bioresponsive scaffold that releases the first therapeutic agent and/or the engineered particle comprising the second therapeutic agent and/or any further encapsulated anti-cancer agent into a tumor microenvironment upon exposure to factors within the microenvironment. In one aspect, the bioresponsive hydrogel comprises a reactive oxygen species (ROS) degradable hydrogel. It is understood and herein contemplated that the release of the first therapeutic agent and/or the engineered particle comprising the second therapeutic agent and/or any further encapsulated anti-cancer agent by the bioresponsive hydrogel into a tumor microenvironment is affected by the microenvironment. In one aspect, disclosed herein are methods of treating, preventing, inhibiting, or reducing a cancer or metastasis in a subject, wherein the hydrogel releases the first therapeutic agent and or the engineered particle into the tumor microenvironment for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

It is understood and herein contemplated that the bioresponsive hydrogel and the engineered particle can be administered separately (concurrent or sequential administration) to the site of a tumor or administered simultaneously by encapsulating the engineered particle in the bioresponsive hydrogel prior to administration. Thus, it is contemplated herein that the bioresponsive hydrogel and engineered particle can be maintained separately and administered concurrently or sequentially. For example, the bioresponsive hydrogel can be administered to the site of the tumor in the subject at least 1, 2, 3, 4, 5, 10, 15, 20, 25, 30, 35, 40, 45, 50, 55 minutes, 1, 2, 3, 4, 5, 6, 7, 8, 9, 10, 11, 12, 18, 24, 36, 48, or 72 hours before the engineered particle. Similarly, the bioresponsive hydrogel can be administered to the site of the tumor in the subject at least 1, 2, 3, 4, 5, 10, 15, 20, 25, 30, 35, 40, 45, 50, 55 minutes, 1, 2, 3, 4, 5, 6, 7, 8, 9, 10, 11, 12, 18, 24, 36, 48, or 72 hours after the engineered particle.

In one aspect, the amount of the pharmaceutical compositions, bioresponsive hydrogels, and/or engineered particles disclosed herein which are administered to the subject for use in the disclosed methods can comprise any amount appropriate for the treatment of the subject for the particular cancer as determined by a physician. For example, the amount of the pharmaceutical compositions, bioresponsive hydrogels, and/or engineered particles can be from about 10 mg/kg to about 100 mg/kg. For example, the amount of the pharmaceutical compositions, bioresponsive hydrogels, and/or engineered particles administered can be at least 10 mg/k, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. Accordingly, in one aspect, disclosed herein are methods of treating a cancer in a subject, wherein the dose of the administered pharmaceutical compositions, bioresponsive hydrogels, and/or engineered particles is from about 10 mg/kg to about 100 mg/kg.

As noted above, it is understood and herein contemplated that the disclosed methods of treating, preventing, inhibiting, or reducing a cancer or metastasis in a subject can further comprise the administration of any anti-cancer agent that would further aid in the reduction, inhibition, treatment, and/or elimination of the cancer or metastasis (such as, for example, gemcitabine). Anti-cancer agents that can be used in the disclosed bioresponsive hydrogels or as an additional therapeutic agent in addition to the disclosed pharmaceutical compositions, engineered particles, and/or bioresponsive hydrogels (including bioresponsive hydrogels that have an engineered particle encapsulated therein) for the methods of reducing, inhibiting, treating, and/or eliminating a cancer or metastasis in a subject disclosed herein can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

The disclosed compositions can be used to treat any disease where uncontrolled cellular proliferation occurs such as cancers and metastasis, including, but not limited to cancers with low PD-L1 expression or a non-immunogenic cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, disclosed herein are methods of inducing blockade inhibitor susceptibility in a tumor in a subject with a cancer comprising administering to the subject any of the bioresponsive hydrogels disclosed herein (such as, for example, a bioresponsive hydrogel comprising a first therapeutic agent and an engineered particle, wherein the engineered particle comprises a second therapeutic agent; and wherein one of the therapeutic agent comprises a hypomethylating agent (HMA) and the other therapeutic agent comprises an immune blockade inhibitor).

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Dual-Bioresponsive Drug Delivery Depot for Combination of Epigenetic Modulation of Immune Checkpoint Blockade

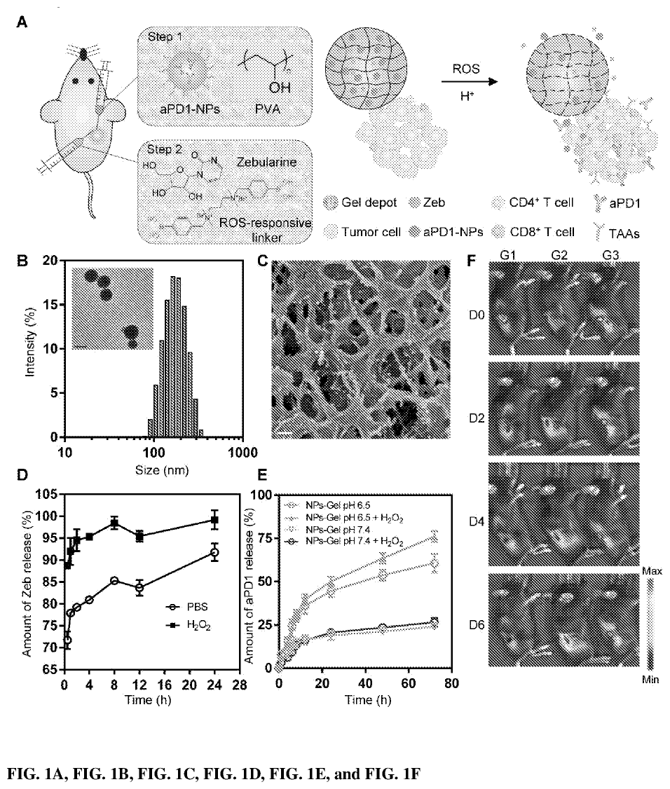

Herein, is described a dual-bioresponsive in situ formed depot to locally co-deliver Zebularine (Zeb), one of demethylation agents, and aPD1 antibody (FIG. 1A). aPD1 antibody was first loaded in the pH-sensitive CaCO₃ nanoparticles (aPD1-NPs) for locally sustained release and then the aPD1-NPs and Zeb were encapsulated together into the ROS-responsive hydrogel (Zeb-aPD1-NPs-Gel) crosslinked by mixing polyvinyl alcohol (PVA) and N¹-(4-boronobenzyl)-N³-(4-boronophenyl)-N¹,N¹,N³,N³-tetramethylpropane-1,3-diaminium (TSPBA) linker. This ROS/H⁺ dual-sensitive scaffold was engineered to utilize the acidic tumor microenvironment and ROS within tumors for the controlled release and increasing retention time of therapeutics. We expected that Zeb-loaded hydrogel could regulate the expression of TAAs, reverse the immunosuppressive tumor microenvironment by reducing suppressive immune cells, and up-regulate PD-L1 expression. Together with the controlled release of aPD1 that blocks PD-L1 interaction with PD-1, the dual responsive depot would elicit strong antitumor immune response.

To select the optimal HMA for the combination therapy, four agents including 5-Azacytidine (AC), 5-Aza-2′-deoxycytidine (DAC), Zebularine (Zeb), and 5-Fluoro-2′-deoxycytidine (5-F) have been tested. AC and DAC are two of the most studied HMAs and have been approved for the treatment of myelodysplastic syndrome (MDS) and acute myeloid leukemia (AML). As a recently studied agent, Zeb, with better stability and lower toxicity, has shown potential for targeting cancer cells preferentially in vitro/vivo. Another pyrimidine nucleoside analog, 5-F, which has a fluorine atom instead of the proton at C5 position, has been studied to reduce the proliferation of brain tumors. First, MTT assays were conducted to compare the cytotoxicity among these agents and determine the appropriate concentration to induce the expression of TAAs. The IC₅₀ values of DAC and 5-F against B16F10 melanoma cells were 2.55 μM and 1.05 μM, respectively, which were much lower than that of AC (48.98 μM) and Zeb (69.18 μM). This high cytotoxicity can significantly impede the application of DAC and 5-F since they directly cause the death of tumor cells rather than inducing TAAs. Thus AC and Zeb with suitable IC₅₀ values were selected for the following study. Next compared was the induction of TAAs between AC and Zeb. Three of TAAs, including melanoma antigen family E1 (MAGE-E1), tyrosinase-related protein-1 (TRP1), and melanoma cell adhesion molecule (CD146) antigens were detected. It was shown that Zeb induced similar or slightly more tumor antigen expression than AC. Furthermore, AC is quite unstable in aqueous solutions while Zeb is stable in neutral and acidic solutions, making potential clinical application of the latter more flexible. Therefore, Zeb was finally chosen as a TAA-inducing agent for further study.

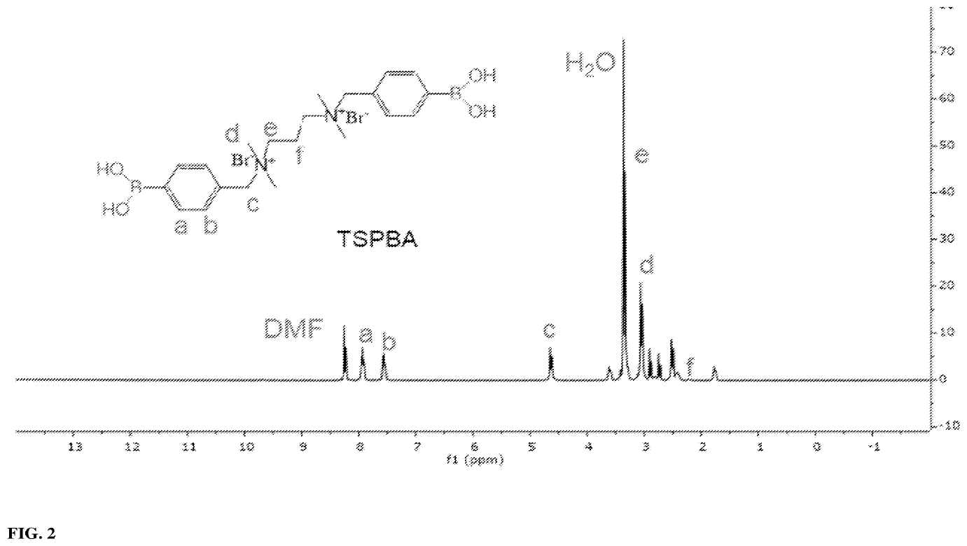

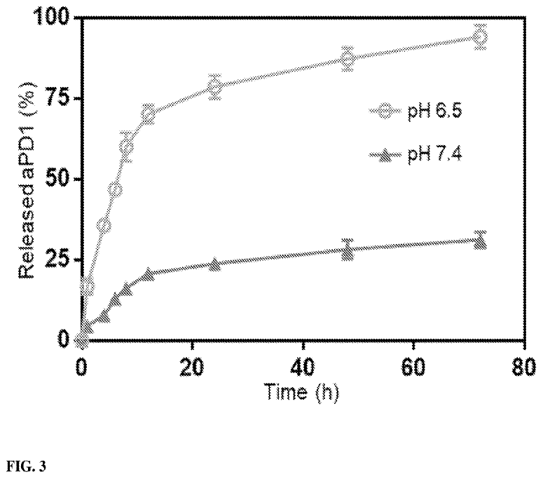

Next, the aPD1-loaded CaCO₃ nanoparticles were prepared using poly(ethylene glycol)-poly(glutamic acid) (PEG-P(Glu)) block copolymers to interact with Ca²⁺ and CO₃²⁻ in the aqueous solution. Glu provided carboxyl to interact with Ca²⁺ preventing the mineralization of large CaCO₃ blocks, and PEG shell acted to avoid agglomeration and aggregation. Monodisperse aPD1-NPs were achieved with an average size of about 100 nm (FIG. 1B) and encapsulation efficiency of about 50%. The ROS-responsive TSPBA linker was synthesized and then characterized by ¹H-NMR (FIG. 2). The hydrogel was immediately formed when mixing the linker with PVA due to the conjugation between the phenylboronic acid and the cis-1,3-diol in PVA. TSPBA linker solution containing Zeb was added into the PVA solution containing aPD1-NPs, leading to the immediate formation of hydrogel, Zeb-aPD1-NPs-Gel. The cryo-scanning electron microscopy (Cyro-SEM) images of this dried scaffold showed that the spherical NPs were loaded in the hydrogel with network structure (FIG. 1C). Additionally, TSPBA linker can be oxidized and hydrolyzed when exposed to H₂O₂, leading to the degradation of the hydrogel and release of Zeb (FIG. 1D). aPD1-NPs can dissolve and release the aPD1 in slightly acidic buffer by reacting with H⁺ (FIG. 3), with almost 95% release amount within 72 h. What's more, encapsulation of aPD1-NPs in the gel depot allowed a more controlled release of aPD1. As shown in FIG. 1E, about 75% of aPD1 antibodies released triggered by both H₂O₂ and low pH stimuli at 72 h. To investigate the retention ability of aPD1 antibodies in vivo, aPD1 labeled with Cyanine5.5 (Cy5.5) was loaded in the gel depot and then peritumorally injected. The fluorescent signal remained detectable six days after gel implantation, while there was nearly no signal for the groups of free Cy5.5-aPD1 and Cy5.5-aPD1-NPs, indicating that encapsulation of aPD1-NPs in gel increased its retention in the tumor sits (FIG. 1F).

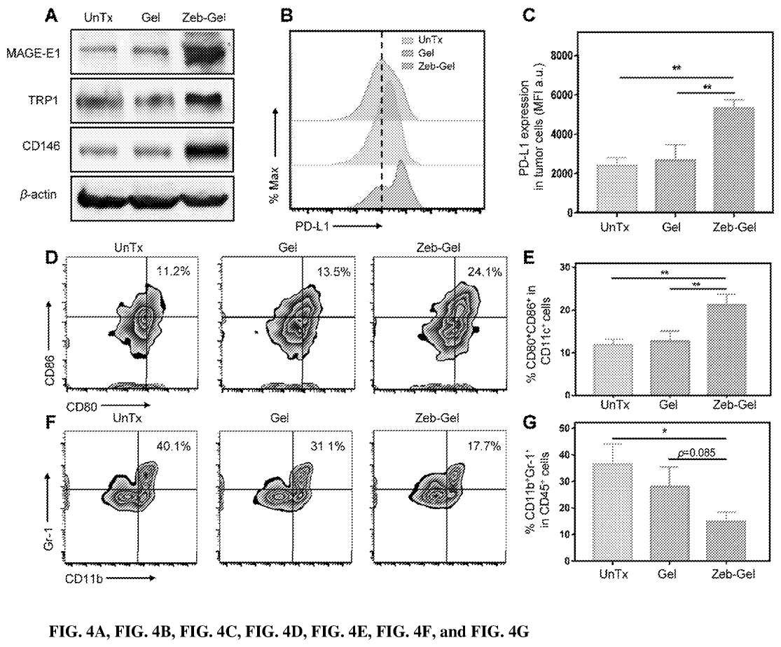

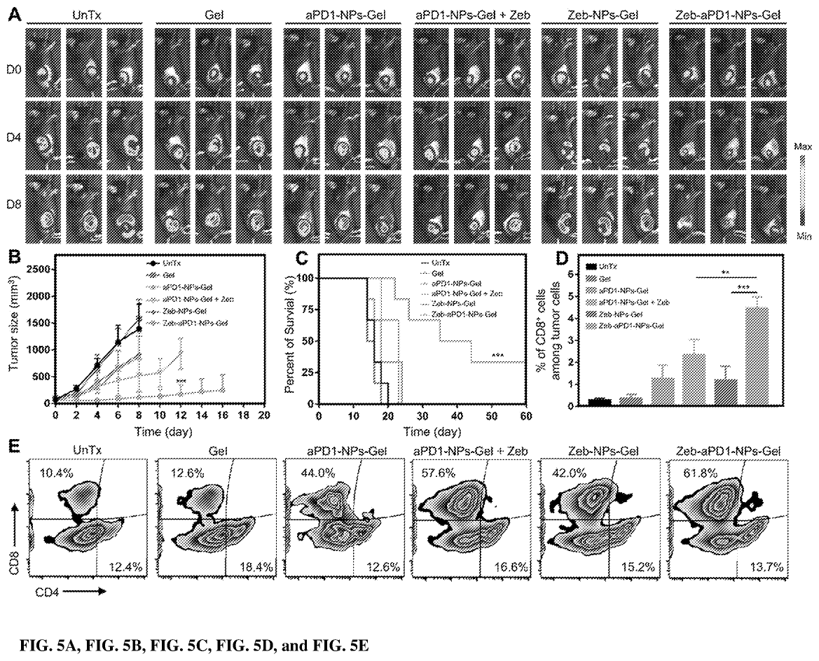

Then, TAAs expression of Zeb-loaded gel (Zeb-Gel) treated group was investigated by western blotting assay. The result showed enhanced expression of MAGE-E1, TRP1, and CD146, indicating that Zeb can facilitate exposure of TAAs and thereby increase the immunogenicity of melanoma, which can potentiate the capture of tumor antigen by antigen presenting cells (FIG. 4A). Treatment with Zeb-Gel also promoted mature dendrictic cells (mDCs) with expression of CD80 and CD86 (FIGS. 4D and 4E). Furthermore, a significant reduction of MDSCs was detected in the Zeb-Gel group, which was about 30% of untreated group (UnTx) and half of Gel treated group, indicating that Zeb can reverse immunosuppressive tumor microenviroment (FIGS. 4F and 4G). Additionally, the presence of Zeb up-regulated the expression of PD-L1 on B16F10 tumors (FIGS. 4B and 4C), making the subsequent use of immune checkpoint blockade essential. To validate whether the combination strategy of epigenetic modulation and ICB therapy can enhance the inhibition of tumor growth, the B16F10 melanoma-bearing mice model was established. Different formulations were injected at the peritumoral site, including blank Gel, aPD1-NPs-Gel (aPD1, 40 μg per mouse), aPD1-NPs-Gel+Zeb (aPD1, 40 μg per mouse; Zeb, 5 mg/kg), Zeb-NPs-Gel (Zeb, 5 mg/kg), and Zeb-aPD1-NPs-Gel (aPD1, 40 μg per mouse; Zeb, 5 mg/kg). The in vivo tumor growth was monitored by capturing bioluminescence images of luciferase-tagged B16F10 cells (FIG. 5A). The Zeb-aPD1-NPs-Gel treated group showed the most notable tumor inhibition effect, while blank Gel treated group had shown negligible treatment efficacy, and the single agent treated groups displayed a limited inhibitory effect on tumor growth (FIG. 5B). Furthermore, the average tumor volume of Zeb-aPD1-NPs-Gel group at day 12 was 4.27-fold smaller than that of aPD1-NPs-Gel+Zeb treated group, which was attributed to the controlled release of Zeb from the gel depot. What's more, the median survival time of mice treated with Zeb-aPD1-NPs-Gel was effectively prolonged to 39.5 days, significantly longer than the other groups (p<0.001), including the untreated group (16 days), blank Gel (15 days), aPD1-NPs-Gel (18 days), Zeb-NPs-Gel (16 days) and aPD1-NPs-Gel+Zeb (23 days) (FIG. 5C). Even one-third of mice treated with Zeb-aPD1-NPs-Gel survived for more than sixty days.

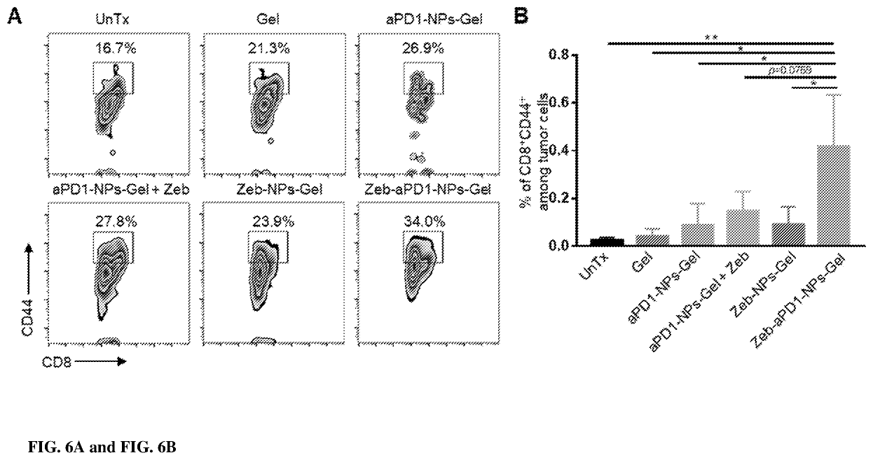

Besides, tumors were harvested for analysis by flow cytometry and immunofluorescence analysis at five days after different treatments. As shown in FIGS. 5D and 5E, the group treated with Zeb-aPD1-NPs-Gel displayed a significantly higher rate of CD8⁺ T cells infiltration in tumor with 4.46% in average, which was 2.73-fold higher than that of Zeb-NPs-Gel group (p<0.001), and 1.90-fold of aPD1-NPs-Gel+Zeb (p<0.01). As shown in FIG. 6, treatment with Zeb-aPD1-NPs-Gel further effectively promote the amount of activated CD8⁺ T cells (CD8⁺CD44⁺ T cells), potentiating CD8⁺ T cell.

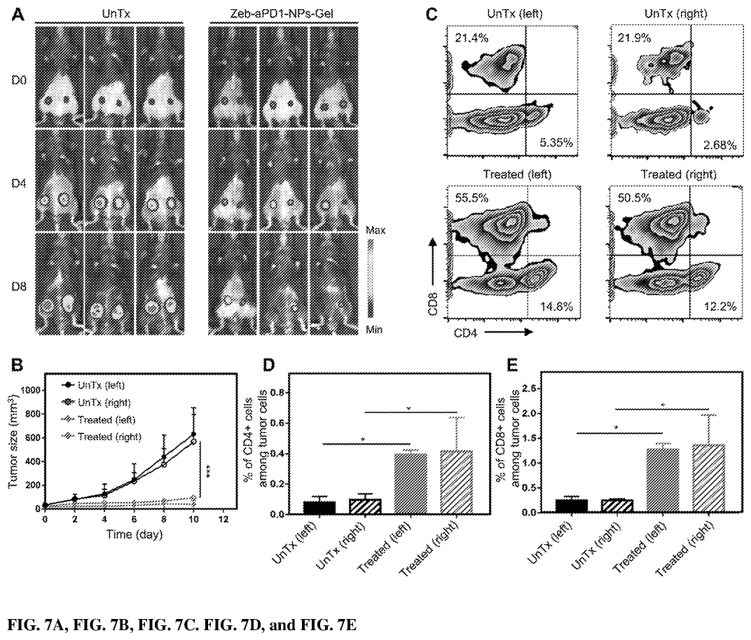

To assess the systemic anti-tumor immune effect of Zeb-aPD1-NPs-Gel, the mice bearing B16F10 tumor on both sides were constructed established. A Zeb-aPD1-NPs-Gel was implanted just next to the left tumor. The tumor growth on both sides had a similar tendency and was obviously inhibited (p<0.001) compared with the control group (FIGS. 7A and 7B). The average tumor volumes of the treated group at the left and right sides ten days after treatment were 14.3-fold and 5.5-fold smaller than that of untreated group on the left side, respectively. In addition, increased infiltration of CD8⁺ T cells and CD4⁺ T cells of tumors (left and right) was detected by flow cytometry compared to the untreated group (p<0.05) (FIG. 7C-7E). These results indicated that local delivery of Zeb-aPD1-NPs-Gel can effectively induce the systemic anti-tumor immune responses.

In summary, a bioresponsive depot loaded with Zeb and aPD1 was engineered to combine epigenetic modulation and immunotherapy, which have been proved to effectively enhance anti-tumor immune responses. This dual-responsive scaffold, composed of pH-sensitive CaCO₃ NPs and ROS-responsive hydrogel, enabled to achieve controlled release of payloads by responding to the acidic pH and ROS condition associated with tumor microenvironment. Additionally, local release of Zeb increased the immunogenicity of tumors via enhancing TAAs expression, decreasing immunosuppression. Based on these functions, its combination with aPD1 inhibitors effectively boosted the T cell-mediated anti-tumor immune response. This delivery strategy integrated with both epigenetic modulators and immune checkpoint blockade treatments can be translated for enhancing objective response rates in clinic.

### a) Materials and Methods

(1) Western Blotting Assay:

The western blotting analysis was performed to investigate the various TAAs expression levels of B16F10 melanoma. For the in vitro study, the B16F10 cells were treated with different demethylation agents at a predetermined concentration for 72 h, and then the drug-loaded medium was removed and replaced by normal dulbecco's modified eagle medium (DMEM) and cells were incubated for another four days. For the in vivo study, the mice bearing melanoma were implanted with Gel or Zeb-Gel for five days. Proteins were collected from cells or tumor tissues using RIPA buffer and the total protein concentrations were quantified using BCA Protein Assay Kit (Thermo Fisher Scientific). Equal amounts of proteins were mixed with 2× laemmli sample buffer, then loaded and separated by Mini-PROTEAN® TGX™ Precast Protein Gel (Bio-Rad) and transferred to a membrane (Bio-Rad), blocked in 3% fat-free milk for 1 h at room temperature, and then incubated with the following primary antibodies diluted in 1.5% bovine serum albumin (BSA) overnight at 4° C.: anti-beta actin antibody, anti-TRP1 antibody, anti-CD146 antibody, and anti-MAGE-E1 antibody. Then, the goat anti-rabbit/mouse HRP-conjugated secondary antibodies were diluted and incubated for 1 h Images were acquired by chemiluminescence.

### (2) Synthesis of ROS-Responsive TSPBA Linker and PEG-P(Glu) Block Copolymers

TSPBA was synthesized from the quaternization reaction between N,N,N′,N′-tetramethyl-1,3-propanediamine (TMPA) and 4-(bromomethyl) phenylboronic acid. Briefly, 4-(bromomethyl) phenylboronic acid and TMPA were mixed (3:1, mmol/mmol) in N,N-Dimethylformamide (DMF) and stirred at 60° C. for 24 h. Then, the reaction solution was precipitated in tetrahydrofuran (THF) and filtrated, and further washed with THF three times. Placing the product under vacuum condition overnight to obtain pure TSPBA, which was then characterized by ¹H-NMR.

The PEG-P(Glu) block copolymers were synthesized. Briefly, a N-carboxyanhydride of γ-benzyl L-glutamate (NCA-BLG) was synthesized by Fuchs-Farthing method using triphosgene and L-Glutamic acid γ-benzyl ester. Then, the PEG-poly(γ-benzyl L-glutamate) (PEG-PBLG) were obtained by following ring-opening procedures in DMF to initiate NCA-BLG by utilizing the primary amino group of CH₃O-PEG-NH₂. Finally, benzyl groups of PEG-PBLG was removed by mixing with 0.5 N NaOH at room temperature to obtain PEG-P(Glu). The repeat unit of the P(Glu) segment of PEG-P(Glu) was calculated to be 50 using 41-NMR spectroscopy (300 MHz; solvent: D20).

### (3) Preparation and Characterization of Zeb-aPD1-NPs-Gel

First, aPD1-NPs were prepared via chemical precipitation. Briefly, 5 mg PEG-P(Glu) was dissolved in DI water and then 80 μg aPD1 was added, followed by the addition of 10 mg CaCl₂ aqueous solution. Then, 1 mM Tris-HCl buffer (pH 8.0) was slowly added to adjust the pH value to pH 7.8 to form Ca²⁺ chelate compounds. And 3 mg Na₂CO₃ was added dropwise to the mixture until opalescence was observed indicating the formulation of aPD1-NPs. The mixture was stirred at 4° C. overnight and then centrifuged to remove the excess ions, copolymers, and antibodies (14,800 rpm, 15 mM). The size distribution was characterized by DLS and the morphology was observed by TEM (JEOL 2000FX). The encapsulation efficiency of aPD1 in CaCO₃ NPs was measured by ELISA (rat IgG total ELISA kit, Abcam, cat. no. ab189578). Then a predetermined amount of Zeb was dissolved in 10 wt % TSPBA solution and then added into the 5 wt % PVA containing aPD1-NPs to form the Zeb-aPD1-NPs-Gel. The morphology of this scaffold was characterized by Cyro-SEM (JEOL 7600F with Gatan Alto).

### (4) In Vitro Release of Zeb and aPD1 from Hydrogel

For the release of Zeb, 1 mM H₂O₂ was added into PBS buffer to investigate the release profile of Zeb from ROS-responsive hydrogels, the amount of Zeb was analyzed by HPLC. For aPD1 release, it was studied in PB buffer with different pH values (pH 6.5 or pH 7.4) with or without H₂O₂. Released aPD1 was measured by a Rat IgG total ELISA kit. All the study was conducted at room temperature.

### (5) In Vivo Anti-Tumor Effect

The C57BL/6 male mice model bearing subcutaneous B16F10 melanoma was established by implanting about 1×10⁶ of luc-B16F10 cells into the right flank of mice. Six days later, the mice were randomly divided into six groups (n=6) and peritumorally implanted with different formulations, including blank Gel, aPD1-NPs-Gel, aPD1-NPs-Gel+Zeb, Zeb-NPs-Gel and Zeb-aPD1-NPs-Gel at a dose of 40 μg aPD1 and/or 5 mg/kg Zeb per mouse. For bioluminescence imaging, 100 μL D-luciferin substrate solution (30 mg/mL) was intraperitoneally injected for 5 min and then the mice were photographed via the IVIS imaging system (Perkin Elmer Ltd). The tumor volumes were measured every two days and calculated according to the equation: (long diameter×short diameter²)/2. Survival time of model mice was recorded starting from the day implanting tumor cells, and Kaplan-Meier survival curves were plotted. Animals were euthanized when showing signs of imperfect health or when the size of tumors exceeded 1.5 cm³.

### (6) Flow Cytometry

Mice model was built and treated as mentioned above. Five days later, mice were euthanized and tumors were collected and homogenized in cold cell staining buffer to obtain single cell suspensions after filtration. Cells were stained with different fluorescence-labeled antibodies following the instructions. The stained cells were measured on a CytoFLEX flow cytometer (Beckman) and analyzed by the FlowJo software.

### (7) In Vivo Systemic Immune Effect on Treating Distant Tumor

1×10⁶ of luc-B16F10 cells were implanted on both sides of mice. Seven days later, Zeb-aPD1-NPs-Gel was injected on the left tumor site, while no treatment was performed on the right tumor site. The in vivo bioluminescence images and tumor volumes on both sides were imaged as aforementioned. Ten days later, the mice were sacrificed and tumors were collected to conduct the flow cytometry experiments aforementioned.

### (8) Statistical Analysis

All results are presented as mean±SD. as indicated and carried out by GraphPad Prism 7.0. Tukey's post-hoc tests and one-way analysis of variance (ANOVA) were performed for multiple comparisons (at least three groups were compared). Survival benefit was determined using the log-rank test. The threshold for statistical significance was p<0.05.

### (9) Chemicals and Reagents

4-(Bromomethyl) phenylboronic acid, N,N,N′,N′-tetramethyl-1,3-propanediamine (TMPA), Polyvinyl alcohol (PVA, 89-98 kDa, 99% hydrolysis), 5-Azacytidine (AC), 5-Aza-2′-deoxycytidine (DAC), Zebularine (Zeb), 5-Fluoro-2′-deoxycytidine (5-F), and L-Glutamic acid γ-benzyl ester were purchased from Sigma-Aldrich. mPEG-Amine (MW 10 kDa) was purchased from Laysan Bio. D-Luciferin-K⁺ salt bioluminescent substrate (Catalog no. NC0921725) was purchased from Perkin Elmer LLC. Anti-PD-L1 antibody, anti-TRP1 antibody (ab178676), and anti-CD146 antibody (ab75769) were obtained from Abcam. Anti-Melanoma antigen family E1 antibody (Novus Biologicals™, Catalog no. NBP191489). Anti-mouse PD-1 antibody (Catalog no. 114114), anti-CD3 antibody (Catalog no. 100204), anti-CD4 antibody (Catalog no. 100412 APC), anti-CD8 antibody (Catalog no. 100707), anti-CD45 antibody (Catalog no. 103108), anti-CD11b antibody (Catalog no. 101208), anti-Gr-1 antibody (Catalog no. 108412), anti-CD11c antibody (Catalog no. 117307), anti-CD80 antibody (Catalog no. 104705), anti-CD86 antibody (Catalog no. 105011), anti-CD8 antibody (Catalog no. 201703), anti-CD44 antibody (Catalog no. 103029), anti-CD3 antibody (Catalog no. 100205), anti-CD4 antibody (Catalog no. 100433), and anti-Foxp3 antibody (Catalog no. 126407) were purchased from Biolegend.

### (10) Mtt Assay

B16F10 cells were cultured into 96-well plates at a density of 5×10³ cells per well overnight. Then the DMEM medium was respectively replaced by DMEM medium containing different concentrations of AC, DAC, Zeb, or 5-F. After incubation for 72 h, 5 mg/mL MTT solution was added to each well for another 4 h incubation. Finally, the rate of cell viability was calculated according to the absorbance at 560 nm by a plate reader (Power Wave XS, Bio-TEK, USA).

### (11) In Vitro Release of aPD1 from CaCO₃ Nanoparticles

The release of aPD1-loaded NPs was conducted in PB buffer with different pH values (pH 6.5 and 7.4) at room temperature. Released aPD1 was measured by a Rat IgG total ELISA kit.

### (12) Cell Lines

The luciferase-tagged B16F10 melanoma cell line (luc-B16F10) was kindly provided by Dr. Leaf Huang at the University of North Carolina at Chapel Hill (UNC-CH). B16F10 cells were maintained with DMEM (Gibco, Invitrogen) supplemented with 10% fetal bovine serum (Invitrogen), penicillin (100 U/ml; Invitrogen), and streptomycin (100 U/ml; Invitrogen).

### (13) Mice

Six- to eight-week-age C57BL/6 male mice were purchased from the Jackson Laboratory and used throughout all experiments. All animal studies were strictly in accordance with the animal protocol approved by the Institutional Animal Care and Use Committee at UNC-CH and North Carolina State University (NCSU). All mice were kept in accordance with federal and state policies on animal research at UNC-CH and NCSU.

### E. References

Gnyszka, Z. Jastrzebski, S. Flis, Anticancer Res. 2013, 33, 2989-2996; bV. E.
Marquez, J. A. Kelley, R. Agbaria, T. Ben-Kasus, J. C. Cheng, C. B. Yoo, P. A. Jones, Ann. N. Y. Acad. Sci. 2005, 1058, 246-254; cD.
Neureiter, S. Zopf, T. Leu, O. Dietze, C. Hauser-Kronberger, E. G. Hahn, C. Herold, M. Ocker, Scand. J. Gastroenterol. 2007, 42, 103-116.
A. R. Karpf, D. A. Jones, Oncogene 2002, 21, 5496; bC.
De Smet, O. De Backer, I. Faraoni, C. Lurquin, F. Brasseur, T. Boon, Proc. Natl. Acad. Sci. U.S.A. 1996, 93, 7149-7153.
E. Héninger, T. E. Krueger, J. M. Lang, Front. Immunol. 2015, 6, 29; bJ.
Yu, M. Ni, J. Xu, H. Zhang, B. Gao, J. Gu, J. Chen, L. Zhang, M. Wu, S. Zhen, Bmc Cancer 2002, 2, 29; cJ.
Yu, H. Zhang, J. Gu, S. Lin, J. Li, W. Lu, Y. Wang, J. Zhu, Bmc cancer 2004, 4, 65; dS.
James, P. Link, A. Karpf, Oncogene 2006, 25, 6975; eM.
Rao, N. Chinnasamy, J. A. Hong, Y. Zhang, M. Zhang, S. Xi, F. Liu, V. E. Marquez, R. A. Morgan, D. S. Schrump, Cancer Res. 2011, 71, 4192.
G. Huls, Blood 2015, 126, 283-284; bJ.
Almasri, H. B. Alkhateeb, B. Firwana, M. B. Sonbol, M. Damlaj, Z. Wang, M. H. Murad, A. Al-Kali, Syst. Rev. 2018, 7, 144; cF. Ferrara, Hematol. Oncol. 2014, 32, 1-9.
J. A. Wargo, P. F. Robbins, Y. Li, Y. Zhao, M. El-Gamil, D. Caragacianu, Z. Zheng, J. A. Hong, S. Downey, D. S. Schrump, Cancer Immunol. Immunother. 2009, 58, 383-394; bA.
A. Toor, K. K. Payne, H. M. Chung, R. T. Sabo, A. F. Hazlett, M. Kmieciak, K. Sanford, D. C. Williams, W. B. Clark, C. H. Roberts, Br. J. Haematol. 2012, 158, 700-711; cA.
Serrano, I. Castro-Vega, M. Redondo, Cancers 2011, 3, 1672-1690; dH.
Li, K. B. Chiappinelli, A. A. Guzzetta, H. Easwaran, R.-W. C. Yen, R. Vatapalli, M. J. Topper, J. Luo, R. M. Connolly, N. S. Azad, Oncotarget 2014, 5, 587.
J. C. Cheng, C. B. Matsen, F. A. Gonzales, W. Ye, S. Greer, V. E. Marquez, P. A. Jones, E. U. Selker, J. Natl. Cancer Inst. 2003, 95, 399-409; bC.
B. Yoo, J. C. Chuang, H. M. Byun, G. Egger, A. S. Yang, L. Dubeau, T. Long, P. W. Laird, V. E. Marquez, P. A. Jones, Cancer Prev. Res. 2008, 1, 233-240.
J. Wang, Y. Ye, J. Yu, A. R. Kahkoska, X. Zhang, C. Wang, W. Sun, R. D. Corder, Z. Chen, S. A. Khan, J. B. Buse, Z. Gu, ACS nano 2018, 12, 2466-2473; bC.
Wang, J. Wang, X. Zhang, S. Yu, D. Wen, Q. Hu, Y. Ye, H. Bomba, X. Hu, Z. Liu, Sci. Trans. Med 2018, 10, eaan3682.
J. Wrangle, W. Wang, A. Koch, H. Easwaran, H. P. Mohammad, F. Vendetti, W. VanCriekinge, T. DeMeyer, Z. Du, P. Parsana, Oncotarget 2013, 4, 2067;
Yang, C. Bueso-Ramos, C. DiNardo, M. R. Estecio, M. Davanlou, Q.-R. Geng, Z. Fang, M. Nguyen, S. Pierce, Y. Wei, Leukemia 2014, 28, 1280; cE. B.
Garon, N. A. Rizvi, R. Hui, N. Leighl, A. S. Balmanoukian, J. P. Eder, A. Patnaik, C. Aggarwal, M. Gubens, L. Horn, N. Engl. J. Med. 2015, 372, 2018-2028; dM. F.
Sanmamed, L. Chen, Cell 2018, 175, 313-326;
I. Daud, J. D. Wolchok, C. Robert, W.-J. Hwu, J. S. Weber, A. Ribas, F. S. Hodi, A. M. Joshua, R. Kefford, P. Hersey, Journal of Clinical Oncology 2016, 34, 4102.
L. Wang, Z. Amoozgar, J. Huang, M. H. Saleh, D. Xing, S. Orsulic, M. S. Goldberg, Cancer Immunol. Res. 2015, 3: 1030 canimm 0073.2015; bP.
L. Triozzi, W. Aldrich, S. Achberger, S. Ponnazhagan, O. Alcazar, Y. Saunthararajah, Cancer Immunology, Immunotherapy 2012, 61, 1441-1450; cK. P. Terracina, L. J. Graham, K. K. Payne, M. H. Manjili, A. Baek, S. R. Damle, H. D. Bear, Cancer Immunology, Immunotherapy 2016, 65, 1061-1073.
M. C. Leslie, Y. J. Zhao, L. B. Lachman, P. Hwu, G. J. Wu, M. Bar-Eli, Gene Ther. 2007, 14, 316-323.
M. M. Gubin, X. Zhang, H. Schuster, E. Caron, J. P. Ward, T. Noguchi, Y. Ivanova, J. Hundal, C. D. Arthur, W.-J. Krebber, Nature 2014, 515, 577; bP. Sharma, S. Hu-Lieskovan, J. A. Wargo, A. Ribas, Cell 2017, 168, 707-723.
M. Morfouace, B. Nimmervoll, N. Boulos, Y. T. Patel, A. Shelat, B. B. Freeman, 3rd, G. W. Robinson, K. Wright, A. Gajjar, C. F. Stewart, R. J. Gilbertson, M. F. Roussel, J. Neurooncol. 2016, 126, 225-234.
N. Nishiyama, S. Okazaki, H. Cabral, M. Miyamoto, Y. Kato, Y. Sugiyama, K. Nishio, Y. Matsumura, K. Kataoka, Cancer Res. 2003, 63, 8977-8983.
P. A. Barker, A. Salehi, J. Neurosci. Res. 2002, 67, 705-712; bM. Sasaki, K. Nakahira, Y. Kawano, H. Katakura, T. Yoshimine, K. Shimizu, S. U. Kim, K. Ikenaka, Cancer Res. 2001, 61, 4809-4814.
P. Bi, A Ramirez-Martinez, H. Li, J. Cannavino, J. R. McAnally, J. M. Shelton, E. Sanchez-Ortiz, R. Bassel-Duby, E. N. Olson, Science 2017, 356, 323-327; bH.
Ruan, X. Chen, C. Xie, B. Li, M. Ying, Y. Liu, M. Zhang, X. Zhang, C. Zhan, W. Lu, W. Lu, ACS Appl. Mater. Interfaces 2017, 9, 17745-17756.
P. Sharma, J. P. Allison, Science 2015, 348, 56-61; bA.
Hoos, Nat. Rev. Drug Discovery 2016, 15, 235; cQ.
Chen, L. Xu, C. Liang, C. Wang, R. Peng, Z. Liu, Nat. Commun. 2016, 7, 13193; dS.
L. Topalian, M. Sznol, D. F. McDermott, H. M. Kluger, R. D. Carvajal, W. H. Sharfman, J. R. Brahmer, D. P. Lawrence, M. B. Atkins, J. D. Powderly, J. Clin. Oncol. 2014, 32, 1020; eN.
Adra, L. Einhorn, S. Althouse, N. Ammakkanavar, D. Musapatika, C. Albany, D. Vaughn, N. Hanna, Annals of Oncology 2017, 29, 209-214; fA.
B. El-Khoueiry, B. Sangro, T. Yau, T. S. Crocenzi, M. Kudo, C. Hsu, T.-Y. Kim, S.-P. Choo, J. Trojan, T. H. Welling 3rd, Lancet 2017, 389, 2492-2502; gB.
Boyerinas, C. Jochems, M. Fantini, C. R. Heery, J. L. Gulley, K. Y. Tsang, J. Schlom, Cancer Immunol. Res. 2015, 3, 1148-1157; hJ.
M. Lee, A. Cimino-Mathews, C. J. Peer, A. Zimmer, S. Lipkowitz, C. M. Annunziata, L. Cao, M. I. Harrell, E. M. Swisher, N. Houston, J. Clin. Oncol. 2017, 35, 2193-2202.
R. F. Wang, P. F. Robbins, Y. Kawakami, X. Q. Kang, S. A. Rosenberg, J. Exp. Med. 1995, 181, 799-804.
R. J. Motzer, B. Escudier, D. F. McDermott, S. George, H J Hammers, S. Srinivas, S. S. Tykodi, J. A. Sosman, G. Procopio, E. R. Plimack, N. Engl. J. Med. 2015, 373, 1803-1813; bH.
E. Ghoneim, Y. Fan, A. Moustaki, H. A. Abdelsamed, P. Dash, P. Dogra, R. Carter, W. Awad, G. Neale, P. G. Thomas, Cell 2017, 170, 142-157. e119; cH.
Ruan, L. Bu, Q. Hu, H. Cheng, W. Lu, Z. Gu, Adv. Healthcare Mater. 2018, 1801099.
S. L. Topalian, C. G. Drake, D. M. Pardoll, Curr. Opin. Immunol. 2012, 24, 207-212; bL. Chen, X. Han, J. Clin. Invest. 2015, 125, 3384-3391.3310.
Y. Kakizawa, K. Kataoka, Langmuir 2002, 18, 4539-4543; bQ.
Chen, C. Wang, X. Zhang, G. Chen, Q. Hu, H. Li, J. Wang, D. Wen, Y. Zhang, Y. Lu, Nat. Nanotechnol. 2019, 14: 89.

1. A bioresponsive hydrogel comprising a first therapeutic agent and an engineered particle, wherein the engineered particle comprises a second therapeutic agent.

2. The bioresponsive hydrogel of claim 1, wherein one of the first therapeutic agent or the second therapeutic agent comprises a blockade inhibitor and the remaining therapeutic agent comprises a hypomethylating agent (HMA).

3. The bioresponsive hydrogel of claim 2, wherein the blockade inhibitor is a PD-1/PD-L1 blockade inhibitor, CTLA-4/B7-1/2 blockade inhibitor, or CD47/SIRPα blockade inhibitor.

4. The method of claim 3, wherein the PD-1/PD-L1 blockade inhibitor is selected from the group consisting of nivolumab, pembrolizumab, pidilizumab, atezolizumab, avelumab, durvalumab, and BMS-936559; wherein the CTLA-4/B7-1/2 blockade inhibitor comprises Ipilimumab; or wherein the CD47/SIRPα blockade inhibitor is selected from the group consisting of Hu5F9-G4, CV1, B6H12, 2D3, CC-90002, and TTI-621.

5. (canceled)

6. (canceled)

7. (canceled)

8. (canceled)

9. The bioresponsive hydrogel of claim 2, wherein the HMA comprises Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine.

10. The bioresponsive hydrogel of claim 1, wherein the bioresponsive hydrogel comprises a bioresponsive scaffold that releases the first therapeutic agent and the engineered particle into a tumor microenvironment upon exposure to factors within the microenvironment.

11. The bioresponsive hydrogel of claim 10, wherein the bioresponsive hydrogel comprises a reactive oxygen species (ROS) degradable hydrogel.

12. The bioresponsive hydrogel of claim 11, wherein the bioresponsive hydrogel comprises crosslinked polyvinyl alcohol (PVA) and N¹-(4-boronobenzyl)-N³-(4-boronophenyl)-N¹,N¹,N³,N³-tetramethylpropane-1,3-diaminium (TSPBA).

13. The bioresponsive hydrogel of claim 1, wherein the engineered particle comprises a pH responsive material.

14. The bioresponsive hydrogel of claim 13, wherein the engineered particles comprise dextran, CaCO₃, chitosan, hyaluronic acid, as well as polymers thereof.

15. A method of treating a cancer in a subject comprising administering to the subject the bioresponsive hydrogel of claim 1.

16. A method of treating a cancer in a subject comprising administering to the subject a bioresponsive hydrogel and an engineered particle; wherein the bioresponsive hydrogel comprises a first therapeutic agent; and wherein the particle comprises a second therapeutic agent.

17. The method of claim 16, wherein one of the first therapeutic agent or the second therapeutic agent comprises a blockade inhibitor and the remaining therapeutic agent comprises a hypomethylating agent (HMA).

18. The method of claim 15, wherein the blockade inhibitor is a PD-1/PD-L1 blockade inhibitor, CTLA-4/B7-1/2 blockade inhibitor, or CD47/SIRPα blockade inhibitor.

19. The method of claim 18, wherein the PD-1/PD-L1 blockade inhibitor is selected from the group consisting of nivolumab, pembrolizumab, pidilizumab, atezolizumab, avelumab, durvalumab, and BMS-936559; wherein the CTLA-4/B7-1/2 blockade inhibitor comprises Ipilimumab; or wherein the CD47/SIRPα blockade inhibitor is selected from the group consisting of Hu5F9-G4, CV1, B6H12, 2D3, CC-90002, and TTI-621.

20. (canceled)

21. (canceled)

22. (canceled)

23. (canceled)

24. The method of claim 17, wherein the HMA comprises Zebularine (Zeb), 5-azacytidine (AC), 5-Aza-2′-deoxycytidine (decitabine, DAC), 5-Fluoro-2′-deoxycytidine (5-F), N-Phthalyl-L-tryptophan; (S)-2-(1,3-dioxoisoindolin-2-yl)-3-(1H-indol-3-yl)propanoic acid (RG-108), guadecitabine (SGI-110), Hydralazine Epigallocatechin Gallate (EGCG), MG98, 5-aza-4′-Thio-2′-Deoxycytidine (Aza-TdC), or procaine.

25. The method of claim 16, wherein the bioresponsive hydrogel comprises a bioresponsive scaffold that releases the first therapeutic agent into a tumor microenvironment upon exposure to factors within the microenvironment.

26. (canceled)

27. The method of claim 16, wherein the particle comprising the second therapeutic agent is encapsulated in the bioresponsive hydrogel.

28. The method of claim 27, wherein the bioresponsive hydrogel comprises a bioresponsive scaffold that releases the first therapeutic agent and the engineered particle comprising the second therapeutic agent into a tumor microenvironment upon exposure to factors within the microenvironment.

29. The method of claim 25, wherein the bioresponsive hydrogel comprises a reactive oxygen species (ROS) degradable hydrogel.

30. The method of claim 25, wherein the hydrogel releases the first therapeutic agent and or the engineered particle into the tumor microenvironment for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

31. (canceled)

32. (canceled)

33. (canceled)

34. (canceled)

35. (canceled)

36. A method of inducing blockade inhibitor susceptibility in a tumor in a subject with a cancer comprising administering to the subject a bioresponsive hydrogel comprising a first therapeutic agent and an engineered particle, wherein the engineered particle comprises a second therapeutic agent; and wherein one of the therapeutic agent comprises a hypomethylating agent (HMA) and the other therapeutic agent comprises an immune blockade inhibitor.
